(19) United States
(12) Patent Application Publication
(10) Pub. No.: US 2010/0082105 A1
(43) Pub. Date: Apr. 1, 2010

(54) HYDROXYAPATITE CERAMIC FOR SPINAL FUSION DEVICE

(75) Inventor: Fuzhai CUI, Beijing (CN)

Correspondence Address: MATTHIAS SCHOLL, 14781 MEMORIAL DRIVE, SUITE 1319, HOUSTON, TX, 77079, US

(73) Assignee: Beijing Allgens Medical Science & Technology Co., Ltd., Beijing (CN)

(21) Appl. No.: 12/553,106

(22) Filed: Sep. 3, 2009

(30) Foreign Application Priority Data
Sep. 4, 2008 (CN) 200810119617.X

(51) Int. Cl. A61F 2/44

(52) U.S. Cl. 623/17.11

(57) ABSTRACT

A spinal fusion cage, has at least hydroxyapatite ceramic, a grain size of the hydroxyapatite ceramic is less than 300 nm. The spinal fusion cage of the invention improves mechanical strength and especially fracture toughness, overcomes brittleness of a conventional hydroxyapatite ceramic, and meets mechanical requirements for the spinal fusion cage. Meanwhile, the spinal fusion cage of the invention maintains biological activity of hydroxyapatite, and facilitates bone fusion with bones within a body.

### CROSS-REFERENCE TO RELATED APPLICATIONS

Pursuant to 35 U.S.C. §119 and the Paris Convention Treaty, this application claims the benefit of Chinese Patent Application No. 200810119617.X filed on Sep. 4, 2008, the contents of which are incorporated herein by reference.

### BACKGROUND OF THE INVENTION

1. Field of the Invention

The invention relates to a biomedical material field, and more particularly to a spinal fusion cage made of hydroxyapatite ceramic.

2. Description of the Related Art

Spinal fusion is an effective method for treating spinal diseases such as spinal tuberculosis, infection, deformity, degenerative disease, and spinal disc injuries. An intervertebral fusion cage acts on bracing intervertebral space, which enables an anterior longitudinal ligament to be in a stress state and to recover an intervertebral height. Moreover, stabling of the intervertebral fusion cage can be facilitated and bone fusion between intervertebral fusion cages can be promoted by contraction of psoas muscles and compression of the intervertebral fusion cage by a patient's weight.

Spinal fusion cages made of hydroxyapatites can enable materials to have required mechanical strength by adjusting and controlling composition of the materials and size of particles. Therefore, this kind of spinal fusion cage is capable of solving problems such as stress shielding and metal debris caused by metal cages without affecting CT and MRI examination, and providing elements such as calcium and phosphorus for repairing bones, which is beneficial for repairing and fusion of the bones. This material can be produced in large volume and effectively solves a problem of autogenous bones.

However, a conventional hydroxyapatite ceramic is a brittle material, in which hydroxyapatite grain has large size (usually greater than 2 microns) and low compressive strength (less than 0.5 GPa), which causes it can only be applied to biological applications under non-bearing conditions such as artificial joint coating, ear bones and so on.

### SUMMARY OF THE INVENTION

In view of the above-described problems, it is one objective of the invention to provide a spinal fusion cage that overcomes a problem of big brittleness with a conventional hydroxyapatite ceramic and features large compressive strength, fracture toughness.

Solution of the invention is as follows:

A spinal fusion cage, comprising hydroxyapatite ceramic, wherein the grain size of the hydroxyapatite ceramic is less than 300 nm.

In a class of this embodiment, it further comprises a dopant having weight 2% less than the total weight.

In a class of this embodiment, the dopant comprises at least one of a fluorin ion, a carbon element, silicon oxide, zirconia, and a strontium ion.

In a class of this embodiment, a grain size of the dopant is less than 500 nm.

In a class of this embodiment, the spinal fusion cage may have different design types and size so as to be adapted for different patients and different parts of damaged spines.

In a class of this embodiment, a grain size of the hydroxyapatite ceramic is less than 190 nm.

In a class of this embodiment, a grain size of the hydroxyapatite ceramic is less than 100 nm.

In a class of this embodiment, the dopant comprises carbon having weight less than 1% of the total weight, and silicon oxide having weight less than 1% of the total weight.

In a class of this embodiment, a grain size of the dopant is less than 400 nm.

In a class of this embodiment, the dopant further comprises zirconia having less than 0.5% of the total weight.

In a class of this embodiment, the dopant comprises a fluorin ion having weight 2% less than the total weight, and a grain size less than 500 nm.

In a class of this embodiment, the dopant comprises silicon oxide having weight 2% less than the total weight, and a grain size less than 500 nm.

In a class of this embodiment, the dopant comprises zirconia having weight 2% less than the total weight, and a grain size less than 500 nm.

In a class of this embodiment, the dopant comprises a strontium ion having weight 2% less than the total weight, and a grain size less than 500 nm.

In a class of this embodiment, it further comprises carbon hydroxyapatite.

In a class of this embodiment, it further comprises fluoride hydroxyapatite.

In a class of this embodiment, it further comprises silicon hydroxyapatite.

In a class of this embodiment, the dopant further comprises calcium fluoride.

In a class of this embodiment, the spinal fusion cage is in the shape of a rectangular cube.

In a class of this embodiment, the spinal fusion cage is in the shape of a wedge cube.

Advantages of the embodiments of the invention are as follow.

The spinal fusion cage of the invention improves mechanical strength and especially fracture toughness, overcomes brittleness of a conventional spinal fusion cage, and meets mechanical requirements for the spinal fusion cage. Meanwhile, the spinal fusion cage of the invention maintains biological activity of hydroxyapatite, and facilitates bone fusion with bones within a body.

### DETAILED DESCRIPTION OF THE EMBODIMENTS

Detailed description of this invention is given below in conjunction with specific embodiments.

### Embodiment 1

A spinal fusion cage comprises hydroxyapatite ceramic having a grain size less than 100 nm and zirconia having weight 0.5% (mass ratio) less than the total weight. Fracture toughness of the spinal fusion cage is up to 1.57 MPam^{1/2}. The spinal fusion cage is in the shape of a cube with a width of 13 mm, a depth of 13 mm, a height of 6 mm, and a thickness of 2.5 mm. The upper and lower surfaces of the spinal fusion cage are of an anatomic structure with an upper occlusal height of 0.5 mm. The spinal fusion cage is applicable to patients with L5/S1 disc herniation.

### Embodiment 2

The structure is almost the same as embodiment 1, except that the fusion cage has an inclination of 5 degrees (the front is higher than the back) with a width of 23 mm, a depth of 13 mm, a height of 13 mm, and a thickness of 2.5 mm. The upper and lower surfaces of the spinal fusion cage are of an anatomic structure with an upper occlusal height of 0.5 mm. The spinal fusion cage comprises hydroxyapatite ceramic having a grain size less than 100 nm, and is applicable to patients with L4/L5 disc herniation.

### Embodiment 3

The structure and size of spinal fusion cage is the same as Embodiment 1. The spinal fusion cage comprises hydroxyapatite ceramic having a grain size less than 190 nm, and is prepared via a two-stage sintering method. Fracture toughness of the spinal fusion cage is 1.92 MPam^{1/2}. The spinal fusion cage is applicable to patients with L5/S1 disc herniation.

### Embodiment

### 4

A spinal fusion cage comprises hydroxyapatite ceramic having a grain size less than 300 nm, and fluorin having weight 2% less than the total weight and a grain size less than 500 nm.

### Embodiment 5

A spinal fusion cage comprises hydroxyapatite ceramic having a grain size less than 300 nm, carbon having a grain size less than 400 nm and weight 1% less than the total weight, and silicon oxide having a grain size less than 400 nm and weight 1% less than the total weight.

### Embodiment 6

A spinal fusion cage comprises hydroxyapatite ceramic having a grain size less than 300 nm, and silicon oxide having weight 2% less than the total weight and a grain size less than 500 nm.

### Embodiment 7

A spinal fusion cage comprises hydroxyapatite ceramic having a grain size less than 300 nm, and zirconia having weight 2% less than the total weight and a grain size less than 500 nm.

### Embodiment 8

A spinal fusion cage comprises hydroxyapatite ceramic having a grain size less than 300 nm, and a strontium ion having weight 2% less than the total weight and a grain size less than 500 nm.

Problems such as stress shielding, metal debris and so on caused by a metal cage can be avoided with the spinal fusion cage of the invention so as to compliant with CT and MRI examination. Moreover, problems of limited bone sources, complications in the bone source areas and so on can be avoided. Also, the acidic products degraded by other biodegradable materials (such as poly-lactic acid) are avoided, and thus improving biological activity and promoting bone fusion.

Hydroxyapatite is a calcium phosphate, namely Hydroxyapatite (HA) that widely exists in a human body, and mainly in bones and teeth, and features good biocompatibility. Hydroxyapatite is essentially a highly staggered polymeric calcium phosphate polymer ceramic, and a molar ratio between a calcium element and a phosphorus element therein is 1.67. In the bones and teeth, calcium and phosphorus mainly exist in a crystal form of colloidal calcium phosphate, and constitute a complex network structure along with collagen protein. Hydroxyapatite can be closely integrated with collagen protein and cells to promote growth of bones and to play an important role in connection between hard tissue and soft tissue. Research indicates that hydroxyapatite can be implanted into defect parts whereby offering support for forming of early capillary and attachment of host bone cells.

The spinal fusion cage of the invention comprises hydroxyapatite ceramic having a grain size less than 300 nm, and is better than a traditional spinal fusion cage in terms of mechanical performance, and especially fracture toughness, which overcomes brittleness of the conventional spinal fusion cage and meets mechanical requirements for the spinal fusion cage. Meanwhile, the spinal fusion cage of the invention maintains biological activity of the hydroxyapatite, which facilitates bone fusion with bones within a body.

In a spinal fusion cage having hydroxyapatite ceramic of a grain size less than 300 nm, a carbon element, fluorin, silicon oxide, zirconia or a strontium ion each having a grain size less than 500 nm can be mixed therein to obtain carbon hydroxyapatite, fluoride hydroxyapatite, and silicon hydroxyapatite that improve hardness and strength of the hydroxyapatite in varying degrees. Fluoride hydroxyapatite with small amount of F⁻ reduces solubility in vivo, improves differentiation, proliferation, and mineralization of bone cells, and enhances integration with bone tissue. The lower the content of the fluoride is, the better the stability is, and the stronger the absorption of the anti-bone marrow is, and the worse the osteoinductivity is compared to HA. Strontium hydroxyapatite with small amount of strontium ion improves the biocompatibility, bone bonding, osteoinductivity, degradation, and biological activity in vivo, and increases osteogenesis, and the overall time of new bone formation. HA containing 0.1% strontium ion features a faster deposition at the surface of the solution without Sr²⁺ than hydroxyapatite without strontium ion. Adding zirconia improves mechanical properties, but reduces biological activity of the hydroxyapatite. A calcium fluoride is added to hydroxyapatite (0H⁻ is replaced by fluoride) so as to form a fluoride hydroxyapatite and to improve thermal stability and sintering properties of HA-Zr0₂, whereby improving mechanical properties of the composite material HA-Zr0₂. The dopant comprises a single component or multiple components, and has weight 2% less than the total weight and a grain size less than 500 nm.

The spinal fusion cages of the invention may have different design types and size so as to be adapted for different patients and different parts of damaged spines. For example, a spinal fusion cage in the shape of a rectangular cube and with a width of 13 mm, a depth of 13 mm, and a height of 7 mm is applicable for cervical vertebrae, and a spinal fusion cage in the shape of a wedge cube and with a width of 25 mm, a depth of 13 mm, and a height of 13 mm is applicable for lumbar vertebrae, and so on.

The spinal fusion cages of the invention have compressive strength greater than 1 GPa, fracture toughness greater than 1.5 MPam^{1/2}.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A spinal fusion cage, comprising hydroxyapatite ceramic, wherein a grain size of said hydroxyapatite ceramic is less than 300 nm.

2. The spinal fusion cage of claim 1, further comprising a dopant having weight 2% less than the total weight.

3. The spinal fusion cage of claim 2, wherein said dopant comprises at least one of a fluoride ion, a carbon element, silicon oxide, zirconia, and a strontium ion.

4. The spinal fusion cage of claim 2, wherein a grain size of said dopant is less than 500 nm.

5. The spinal fusion cage of claim 1, wherein design types and size of said spinal fusion cage are adapted for different patients and different parts of damaged spines

6. The spinal fusion cage of claim 1, wherein a grain size of said hydroxyapatite ceramic is less than 190 nm.

7. The spinal fusion cage of claim 1, wherein a grain size of said hydroxyapatite ceramic is less than 100 nm.

8. The spinal fusion cage of claim 3, wherein said dopant comprises carbon having weight less than 1% of the total weight, and silicon oxide having weight less than 1% of the total weight.

9. The spinal fusion cage of claim 8, wherein a grain size of said dopant is less than 400 nm.

10. The spinal fusion cage of claim 7, wherein said dopant further comprises zirconia having weight less than 0.5% of the total weight.

11. The spinal fusion cage of claim 4, wherein said dopant comprises a fluorin ion having weight 2% less than the total weight, and a grain size less than 500 nm.

12. The spinal fusion cage of claim 4, wherein said dopant comprises silicon oxide having weight 2% less than the total weight, and a grain size less than 500 nm.

13. The spinal fusion cage of claim 4, wherein said dopant comprises zirconia having weight 2% less than the total weight, and a grain size less than 500 nm.

14. The spinal fusion cage of claim 4, wherein said dopant comprises a strontium ion having weight 2% less than the total weight, and a grain size less than 500 nm.

15. The spinal fusion cage of claim 3, further comprising carbon hydroxyapatite.

16. The spinal fusion cage of claim 3, further comprising fluoride hydroxyapatite.

17. The spinal fusion cage of claim 3, further comprising silicon hydroxyapatite.

18. The spinal fusion cage of claim 3, wherein said dopant further comprises calcium fluoride.

19. The spinal fusion cage of claim 5, wherein design types and size of said spinal fusion cage are adapted for different patients and different parts of damaged spines

20. The spinal fusion cage of claim 5, wherein said spinal fusion cage is in the shape of a wedge cube.
